(19) United States
(12) Patent Application Publication
(10) Pub. No.: US 2024/0062913 A1
(43) Pub. Date: Feb. 22, 2024

(54) METHOD FOR PREDICTING THE OCCURRENCE OF POSTOPERATIVE ACUTE KIDNEY INJURY AND SYSTEM THEREOF

(71) Applicant: THE CATHOLIC UNIVERSITY OF KOREA INDUSTRY-ACADEMIC COOPERATION FOUNDATION, Seoul (KR)

(72) Inventors: Hye Eun YOON, Seongnam-si (KR); Ji Won MIN, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/450,703

(22) Filed: Aug. 16, 2023

(30) Foreign Application Priority Data
Aug. 16, 2022 (KR) 10-2022-0102129

(51) Int. Cl. G16H 50/30; G06N 20/00

(52) U.S. Cl. CPC G16H 50/30; G06N 20/00

(57) ABSTRACT

Disclosed herein are a method and system for predicting an occurrence of acute kidney injury. A method of predicting an occurrence of acute kidney injury, according to some embodiments of the present disclosure, may train a model for predicting a risk of an occurrence of postoperative acute kidney injury using a dataset of a plurality of patients, and accurately and early predict the risk of an occurrence of postoperative acute kidney injury for a specific patient using the trained model.

### CROSS-REFERENCE TO RELATED APPLICATIONS

This application is based on and claims priority from Korean Patent Application No. 10-2022-0102129, filed on Aug. 16, 2022, with the Korean Intellectual Property Office, the disclosure of which is incorporated herein in its entirety by reference.

### TECHNICAL FIELD

The present disclosure relates to a method and system for predicting an occurrence of postoperative acute kidney injury, and more particularly, to a method for predicting a risk of occurrence of postoperative acute kidney injury using machine learning/deep learning technology, and a system for performing the method.

### BACKGROUND

Acute Kidney Injury (AKI) is statistically known to occur in about 7% of all hospitalized patients, and up to 20% of patients treated in the Intensive Care Unit (ICU). In addition, acute kidney injury is known to occur in up to about 40% of patients who undergo surgery.

In particular, acute kidney injury that occurs after surgery drastically reduces the survival rate of patients, and when patients do not recover from acute kidney injury and receive renal replacement therapy, the quality of life of patients who survive is significantly reduced even if the patients survive.

Therefore, there is currently ongoing research in the medical field on a method of predicting early the risk of occurrence of acute kidney injury after surgery. However, since acute kidney injury after surgery is caused by a complex set of factors, early prediction is quite difficult, and the results of research are still insignificant.

[Document of Related Art]

[Patent Document]
Korean Patent Application Laid-Open No. 10-2022-0075046 (published on Jun. 7, 2022)

### SUMMARY

The present disclosure has been made in an effort to provide a method of accurately and early predicting a risk of an occurrence of postoperative acute kidney injury, and a system for performing the method.

The present disclosure has been made in another effort to provide a method of building a model to predict a risk of an occurrence of postoperative acute kidney injury, and a system for performing the method.

The present disclosure has been made in still another effort to provide a method of generating a high-quality dataset for a model that predicts a risk of an occurrence of postoperative acute kidney injury, and a system for performing the method.

The present disclosure has been made in yet another effort to provide key variables (features) that can ensure performance of a model that predicts a risk of an occurrence of postoperative acute kidney injury.

Technical problems to be solved by the present disclosure are not limited to the above-mentioned technical problems, and other technical problems, which are not mentioned above, may be clearly understood from the following descriptions by those skilled in the art to which the present disclosure pertains.

An exemplary embodiment of the present disclosure provides a method of predicting an occurrence of postoperative acute kidney injury, which is performed by at least one computing device, the method may include: preparing a dataset of a plurality of patients—in which a dependent variable of the dataset relates to an occurrence of postoperative acute kidney injury, and independent variables of the dataset include variables relating to preoperative examination items of the patients—; and building a model configured to predict a risk of the occurrence of postoperative acute kidney injury using the prepared dataset.

In some embodiments, the preoperative examination items may include albumin, creatinine (Cr), potassium, protein, and urinary specific gravity.

In some embodiments, the independent variables of the dataset may further include variables relating to disease history and medication history of the patients, in which the disease history may include history regarding chronic kidney disease (CKD), hypertension (HTN), cardiovascular disease (CVD), chronic obstructive pulmonary disease (COPD), and liver cirrhosis (LC). In this case, the medication history may relate to an antihypertensive medication.

In some embodiments, the independent variables of the dataset may further include variables regarding types and duration of surgeries undergone by the patients.

In some embodiments, the model may be based on at least one of a neural network, logistic regression, and a light gradient boosting machine (LGBM).

In some embodiments, the preparing of the dataset may include removing a patient data satisfying a predetermined kidney-related condition from an original patient dataset.

In some embodiments, the preparing of the dataset may include removing patient data satisfying a predetermined surgery-related condition from an original patient dataset, and in which the predetermined surgery-related condition may be defined based on duration of surgery or types of surgeries.

Another exemplary embodiment of the present disclosure provides a method of predicting an occurrence of postoperative acute kidney injury, which is performed by at least one computing device, the method may include: acquiring a model trained to predict a risk of an occurrence of postoperative acute kidney injury—in which the model is trained using a dataset of a plurality of patients, a dependent variable of the dataset relates to the occurrence of postoperative acute kidney injury, and independent variables of the dataset include variables relating to preoperative examination items of the patients; and predicting a risk of an occurrence of acute kidney injury to a specific patient after a target surgery using the trained model.

Yet another exemplary embodiment of the present disclosure provides a system for predicting an occurrence of postoperative acute kidney injury, the system may include: one or more processors; and a memory configured to store one or more instructions, in which the one or more processors may perform preparing, by executing the stored one or more instructions, a dataset of a plurality of patients—in which a dependent variable of the dataset relates to an occurrence of postoperative acute kidney injury, and independent variables of the dataset include variables relating to preoperative examination items of the patients—; and building a model configured to predict a risk of the occurrence of postoperative acute kidney injury using the prepared dataset.

Yet another exemplary embodiment of the present disclosure provides a system for predicting an occurrence of acute kidney injury, the system may include: one or more processors; and a memory configured to store one or more instructions, in which the one or more processors may perform acquiring, by executing the stored one or more instructions, a model trained to predict a risk of an occurrence of postoperative acute kidney injury—in which the model is trained using a dataset of a plurality of patients, a dependent variable of the dataset relates to the occurrence of postoperative acute kidney injury, and independent variables of the dataset include variables relating to preoperative examination items of the patients—; and predicting a risk of an occurrence of acute kidney injury to a patient after a target surgery using the trained model.

Yet another exemplary embodiment of the present disclosure provides a computer program, the computer program may be stored on a computer-readable storage medium, in conjunction with a computing device, for an execution of preparing a dataset of a plurality of patients—in which a dependent variable of the dataset relates to an occurrence of postoperative acute kidney injury, and independent variables of the dataset include variables relating to preoperative examination items of the patients—and building a model to predict a risk of the occurrence of postoperative acute kidney injury using the prepared dataset.

Yet another exemplary embodiment of the present disclosure provides a computer program, the computer program may be stored on a computer-readable storage medium, in conjunction with a computing device, for an execution of acquiring a model trained to predict a risk of an occurrence of postoperative acute kidney injury—in which the model is trained using a dataset of a plurality of patients, a dependent variable of the dataset relates to the occurrence of postoperative acute kidney injury, and independent variables of the dataset include variables relating to preoperative examination items of the patients; and predicting a risk of an occurrence of acute kidney injury to a specific patient after a target surgery using the trained model.

According to the exemplary embodiments of the present disclosure, a risk of an occurrence of postoperative acute kidney injury can be predicted early and accurately using machine learning/deep learning models (hereinafter, referred to as a predictive model). For example, a risk that acute kidney injury will occur in a patient after surgery may be predicted early and accurately, and an occurrence rate of acute kidney injury may be reduced through proactive actions based on the prediction results.

In addition, a high-quality training dataset for a predictive model can be generated by removing unnecessary patient data from an original patient dataset. Accordingly, a high-performance predictive model can be easily built.

In addition, independent variables of a patient dataset may consist of variables regarding disease history, medication history, surgery-related information, and preoperative/postoperative examination items of a patient. Therefore, the predictive model can be trained to predict a risk of an occurrence of acute kidney injury by considering a variety of factors in a complex manner, and performance of the predictive model can be further improved.

The effects according to the technical spirit of the present disclosure are not limited to the aforementioned effects, and other effects, which are not mentioned above, will be clearly understood by those skilled in the art from the following description.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is an exemplary view for schematically describing a system for predicting the occurrence of postoperative acute kidney injury and an input and output thereof, according to some embodiments of the present disclosure.

FIGS. 2 and 3 are exemplary views for describing a process by which the system for predicting the occurrence of postoperative acute kidney injury provides a prediction service, according to some embodiments of the present disclosure.

FIGS. 4 and 5 are exemplary flowcharts illustrating a method of predicting the occurrence of postoperative acute kidney injury, according to some embodiments of the present disclosure.

FIG. 6 is an exemplary view illustrating a predictive model based on an artificial neural network, according to some embodiments of the present disclosure.

FIG. 7 is an exemplary view for describing a method of selecting key independent variables according to some embodiments of the present disclosure.

FIG. 8 is an exemplary view for describing a method of selecting key independent variables according to some other embodiments of the present disclosure.

FIGS. 9 and 10 are exemplary views for describing a method of augmenting a dataset according to some embodiments of the present disclosure.

FIG. 11 is a view illustrating a process of preparing a patient dataset for performance evaluation of a predictive model.

FIGS. 12 to 14 illustrate performance evaluation results for different types of predictive models.

FIG. 15 illustrates an exemplary computing device capable of implementing the system for predicting the occurrence of postoperative acute kidney injury, according to some embodiments of the present disclosure.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, exemplary embodiments of the present disclosure will be described in detail with reference to the accompanying drawings. Advantages and features of the present disclosure and methods of achieving the advantages and features will be clear with reference to embodiments described in detail below together with the accompanying drawings. However, the technical spirit of the present disclosure is not limited to the exemplary embodiments disclosed below but will be implemented in various forms. The exemplary embodiments of the present disclosure are provided so that the technical spirit of the present disclosure is completely disclosed, and a person with ordinary skill in the art can fully understand the scope of the present disclosure. The technical spirit of the present disclosure will be defined only by the scope of the appended claims.

In giving reference numerals to constituent elements of the respective drawings, it should be noted that the same constituent elements will be designated by the same reference numerals, if possible, even though the constituent elements are illustrated in different drawings. In addition, in the description of the present disclosure, the specific descriptions of publicly known related configurations or functions will be omitted when it is determined that the specific descriptions may obscure the subject matter of the present disclosure.

Unless otherwise defined, all terms (including technical and scientific terms) used in the present specification may be used as the meaning which may be commonly understood by the person with ordinary skill in the art, to which the present disclosure belongs. In addition, terms defined in a generally used dictionary shall not be construed in ideal or excessively formal meanings unless they are clearly and specially defined in the present specification. The terms used in the present specification are for explaining the exemplary embodiments, not for limiting the present disclosure. Unless particularly stated otherwise in the present specification, a singular form also includes a plural form.

In addition, the terms such as first, second, A, B, (a), and (b) may be used to describe constituent elements of the present disclosure. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” between the constituent elements.

The terms “comprise (include)” and/or “comprising (including)” used in the present disclosure are intended to specify the presence of the mentioned constituent elements, steps, operations, and/or elements, but do not exclude presence or addition of one or more other constituent elements, steps, operations, and/or elements.

Hereinafter, various embodiments of the present disclosure will be described in detail with reference to the accompanying drawings.

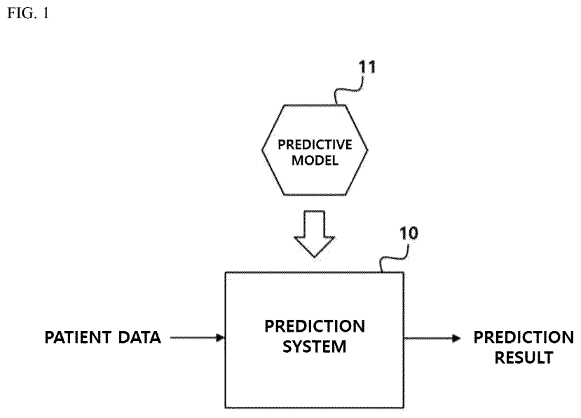

FIG. 1 is an exemplary view for describing a system 10 for predicting the occurrence of postoperative acute kidney injury and an input and output thereof, according to some embodiments of the present disclosure. In the drawings below FIG. 1, a system 10 for predicting the occurrence of postoperative acute kidney injury is illustrated as a prediction system 10, and in the following description, the system 10 for predicting the occurrence of postoperative acute kidney injury will be abbreviated as the prediction system 10.

As shown in FIG. 1, the prediction system 10 may be a computing device/system that predicts and outputs a risk of occurrence of postoperative acute kidney injury (AKI) of a corresponding patient based on input patient data. For example, the prediction system 10 may predict (a risk of occurrence of) acute kidney injury after surgery in a patient who has undergone (or is scheduled to undergo) surgery through a trained predictive model 11 (e.g., (a risk of occurrence of) acute kidney injury within about 30 days after surgery).

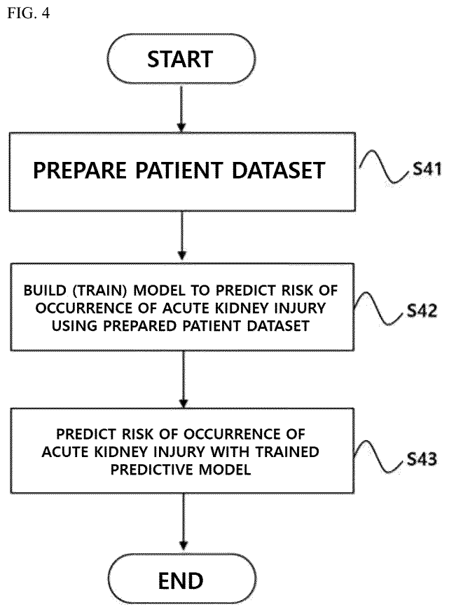

More specifically, the prediction system 10 may train the predictive model 11 using a dataset of a plurality of patients, and predict a risk of occurrence of postoperative acute kidney injury for a specific patient using the trained predictive model 11. Prediction results may include, for example, but are not limited to, whether postoperative acute kidney injury will occur, a risk of acute kidney injury (i.e., the probability that acute kidney injury will occur), a stage of progression/risk of acute kidney injury (e.g., a stage of acute kidney injury according to KDIGO guidelines), and a risk for each stage. A specific method by which the prediction system 10 trains the predictive model 11 and predicts a risk of occurrence of postoperative acute kidney injury will be described in detail with reference to the drawings below FIG. 4.

A patient dataset (or data) used to train (or predict) the predictive model 11 may include at least one dependent variable and a plurality of independent variables, which will be described below. For reference, the term of a variable may be used interchangeably with the terms of a feature, attribute, element, item, and field in the art. In addition, each individual data that constitutes a patient dataset may be used interchangeably with the terms of a sample, example, record, instance, entry, data point, and observation.

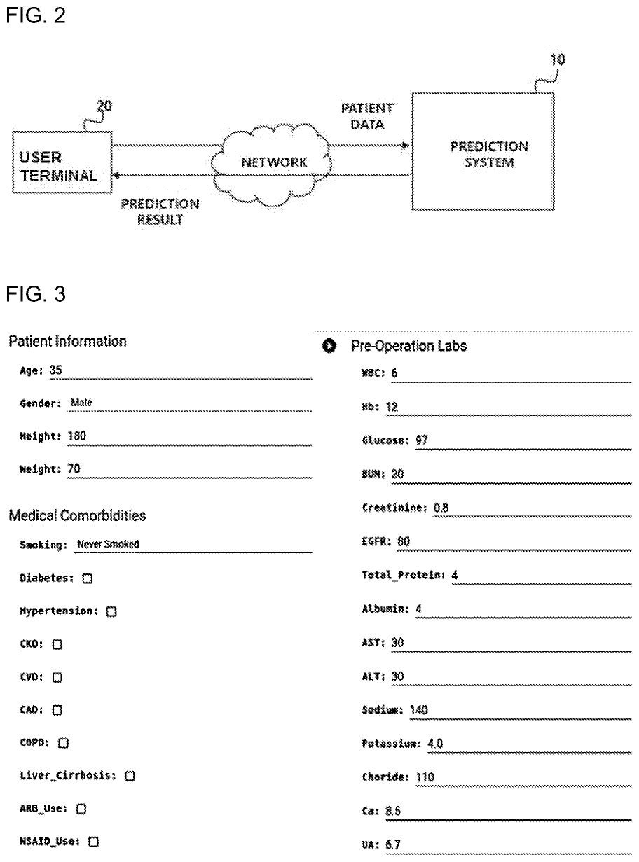

In some embodiments, as illustrated in FIG. 2, the prediction system 10 may provide a prediction service to a user regarding the occurrence of postoperative acute kidney injury. For example, the prediction system 10 may receive patient data from a user terminal 20, predict a risk of the occurrence of postoperative acute kidney injury based on the received patient data, and provide the prediction results to the user terminal 20. The user may be a patient or healthcare provider, but the scope of the present disclosure is not limited thereto. In a more specific example, the prediction system 10 may provide a prediction service through a web interface (or app interface). For example, the prediction system 10 may provide a web page 30, as illustrated in FIG. 3, to the user terminal 20, receive patient data as input through the web page 30, and provide predicted results through the web page 30 based on the input patient data.

The prediction system 10 may be implemented as at least one computing device. For example, the prediction system 10 may be implemented on a single computing device. In another example, the prediction system 10 may be implemented on a plurality of computing devices, such that a first function of the prediction system 10 is implemented on a first computing device and a second function is implemented on a second computing device. Alternatively, a specific function of the prediction system 10 may be implemented on a plurality of computing devices.

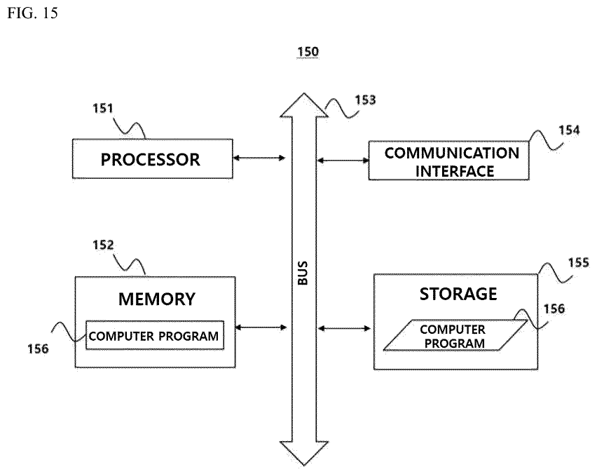

The computing device may include any device having computing (processing) functions, and reference is made to FIG. 15 for an example of such a device. Since the computing device is a collection in which multiple components (e.g., a processor, memory, etc.) interact, the computing device may be referred to as a computing system depending on the case. In addition, the computing system may mean a collection in which a plurality of computing devices interact for the same purpose.

With reference now to FIGS. 1 to 3, the prediction system 10 has been schematically described according to some embodiments of the present disclosure. Hereinafter, various methods that may be performed in the prediction system 10 illustrated in FIG. 1 will be described in detail.

Hereinafter, for convenience of understanding, all steps (operations) of the methods to be described below will be described assuming that all steps are performed in the prediction system 10 illustrated in FIG. 1. Therefore, when a subject of a specific step (operation) is omitted, it may be understood to be performed by the prediction system 10. Of course, in the actual practice, some of the steps of the method described below may be performed on other computing devices. For example, training of the predictive model (e.g., 11 in FIG. 1) may in some cases be performed on a different computing device.

FIG. 4 is an exemplary flowchart schematically illustrating a method of predicting the occurrence of postoperative acute kidney injury, according to some embodiments of the present disclosure. However, it is understood that this is only a preferred embodiment to accomplish the purposes of the present disclosure, and that some steps may be added or omitted as necessary.

As illustrated in FIG. 4, a prediction method according to embodiments may start with step S41 of preparing a patient dataset to be used for training the predictive model. As described above, the patient dataset may include one or more dependent variables and a plurality of independent variables, and may include a plurality of patient data (i.e., data samples).

One or more dependent variables (e.g., a correct answer label) may relate to the occurrence of postoperative acute kidney injury (e.g., the occurrence of acute kidney injury within 30 days after surgery), for example, but not limited to, the occurrence of postoperative acute kidney injury, the progression/risk stage of acute kidney injury (e.g., acute kidney injury stage according to KDIGO guidelines), and the like.

The plurality of independent variables may include, for example, variables regarding the patient's demographic characteristics, disease history, medication history, surgeries the patient has undergone, examination items the patient has undergone before and after surgery (i.e., items that indicate the patient's health state), and the like. However, the scope of the present disclosure is not limited thereto. For a more detailed example of the independent variables, reference is made to Table 1 below.

TABLE 1

| Classification | | Detailed Variables |
|---|---|---|
| Demographics | | Age, gender, BMI, height, weight, blood |
| | | pressure (SBP, DBP), etc. |
| Disease history | | Chronic kidney disease (CKD), diabetes |
| | | mellitus (DM), hypertension (HTN), |
| | | cardiovascular disease (CVD), coronary |
| | | artery disease (CAD), chronic obstructive |
| | | pulmonary disease (COPD), liver cirrhosis |
| | | (LC), smoking status, duration of smoking, |
| | | duration of disease, etc. |
| Medication history | | Antihypertensive medication |
| | | (e.g., angiotensin receptor blocker (ARB), |
| | | angiotensin-converting enzyme inhibitor |
| | | (ACEi), anti-inflammatory medication |
| | | (e.g., non-steroidal anti-inflammatory |
| | | drugs (NSAIDs)), duration of medication, |
| | | dosage, etc. |
| Surgery | | Surgery section, duration of surgery, |
| | | weekday or weekend of surgery date, etc. |
| Examination | Preoperative | White blood cell count (WBC), hemoglobin, |
| items | | C-reactive protein (CRP), glucose, blood |
| | | urea nitrogen (BUN), creatinine (Cr), eGFR, |
| | | protein, total protein, albumin, AST, ALT, |
| | | sodium (Na), potassium (K), chloride (Cl), |
| | | calcium (Ca), uric acid, CPK, LDH, urinary |
| | | specific gravity (SG), and urine protein. |

By using various independent variables as described above to train the predictive model, the predictive model will be able to accurately predict a risk of the occurrence of postoperative acute kidney injury by considering various factors in a complex manner. For example, the predictive model trained using the independent variables exemplified in Table 1 will be able to accurately predict a risk of the occurrence of postoperative acute kidney injury (e.g., a risk of the occurrence of acute kidney injury within 30 days after surgery) by considering the patient's disease history, information on the surgery the patient has undergone (or is scheduled to undergo), and the patient's preoperative/postoperative states in a complex manner.

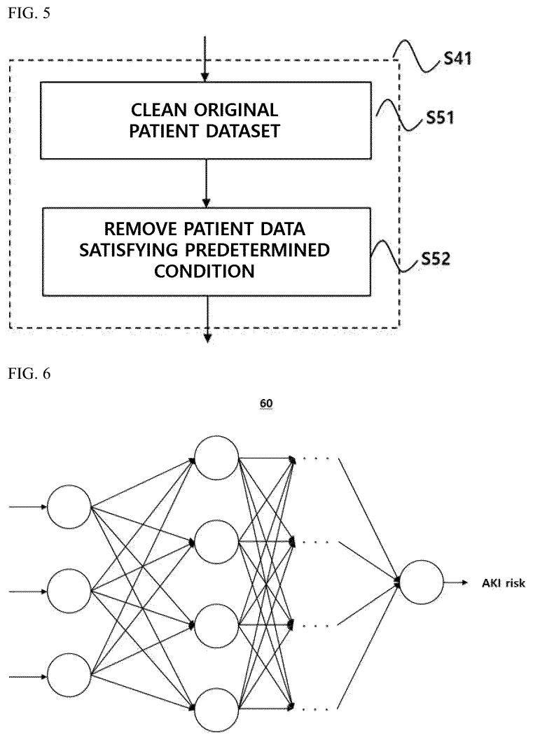

The detailed process of step S41 is illustrated in FIG. 5.

As illustrated in FIG. 5, step S41 of preparing a patient dataset may include step S51 of cleaning an original patient dataset and step S52 of removing some patient data (i.e., data samples). FIG. 5 illustrates an example in which step S52 is performed after step S51, but the order in which step S51 and step S52 are performed may vary. Hereinafter, each step will be described in more detail.

At step S51, the original patient dataset may be cleaned in various ways.

In an example, the prediction system 10 may correct for outliers in the original patient dataset. For example, the prediction system 10 may determine that values in the top n % (e.g., 1%, 5%, etc.) and/or bottom k % (e.g., 1%, 5%, etc.) for each variable are outliers, and remove patient data containing the outliers.

In another example, the prediction system 10 may correct for missing values in the original patient dataset. For example, the prediction system 10 may use multiple imputation by chained equations (MICE) to compensate for (i.e., fill in) missing values in the original patient dataset. The MICE technique is already well known to those skilled in the art, the description of which will be omitted.

In still another example, the prediction system 10 may convert values of non-numeric variables in the original patient dataset to values of numeric variables. For example, the prediction system 10 may convert values of non-numeric variables to values of numeric variables using a one-hot encoding technique.

In still another example, the prediction system 10 may normalize the original patient dataset (or values of numerical variables). For example, the prediction system 10 may normalize the original patient dataset (or values of numerical variables) using a min-max normalization technique.

In another example, the prediction system 10 may clean the original patient dataset based on various combinations of the examples described above.

At step S52, patient data satisfying the predetermined conditions may be removed from the original patient dataset. However, the specific methods may vary depending on the embodiment.

In some embodiments, patient data satisfying a predetermined kidney-related condition may be removed. Here, the predetermined kidney-related condition may be a condition defined based on, for example, a history of renal replacement therapy, a preoperative eGFR level, a preoperative creatinine (Cr) level, or a degree of elevation of Cr level within a period of time prior to surgery. However, the scope of the present disclosure is not limited by these examples. In a more specific example, the prediction system 10 may remove data, which relate to a patient who has a history of receiving renal replacement therapy, a patient who has a preoperative eGFR level below a reference value (e.g., about 15 ml per minute), a patient who has a preoperative creatinine (Cr) level (concentration) above a reference value (e.g., about 4.0 mg/dL), a patient whose preoperative creatinine (Cr) level has increased above a reference value (e.g., about 1.5 times the previous value or 0.3 mg/dL) within a predetermined period of time (e.g., about two weeks), or the like from the original patient dataset. The reason for removing the data for these patients is because the exemplified patients already have chronic kidney disease stage 5, which means the patients have serious kidney problems or have recently suffered from acute kidney injury. That is, it can be understood to remove data from the exemplified patients because it is important to accurately predict postoperative acute kidney injury that occurs suddenly in the general population of patients.

In some other embodiments, patient data satisfying predetermined surgery-related conditions may be removed. Here, the predetermined surgery-related conditions may be conditions defined based on, for example, duration of surgery or type of surgery. However, the scope of the present disclosure is not limited by these examples. In a more specific example, the prediction system 10 may remove data for patients whose duration of surgery is below a reference value (e.g., about 1 hour). This is because the association between relatively simple surgeries performed over a short period of time and acute kidney injury is usually very low. In another example, the prediction system 10 may remove data from patients whose type of surgery corresponds to cardiac surgery, nephrectomy, or kidney transplant. It can also be understood that it is important to accurately predict acute kidney injury, which occurs suddenly in the general population of patients.

In still other embodiments, some patient data may be removed from the original patient dataset based on various combinations of the embodiments described above.

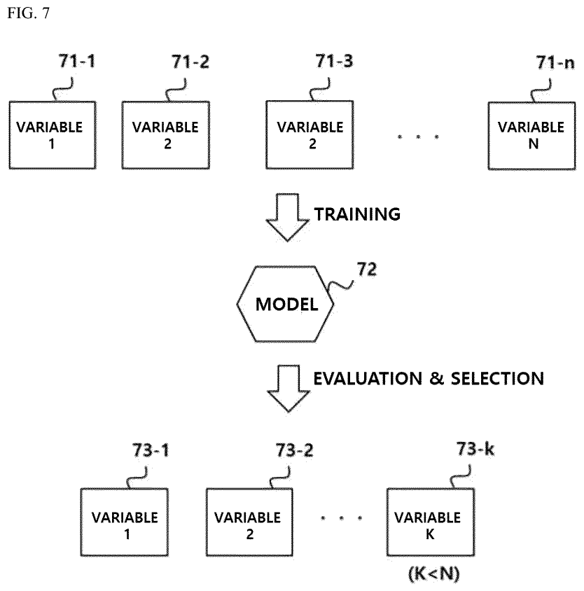

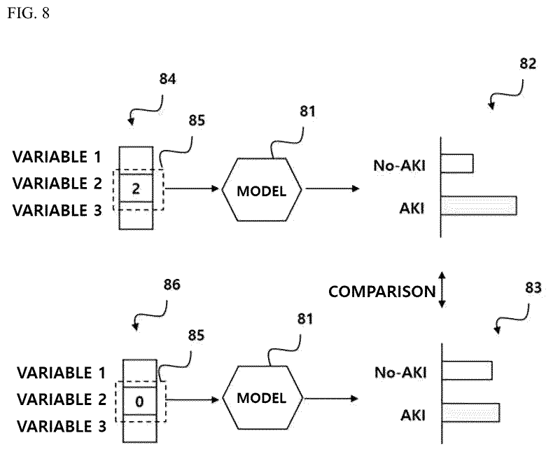

Meanwhile, in some embodiments, key independent variables may be selected from a plurality of independent variables constituting the original patient dataset (or a patient dataset). Further, the patient dataset for the key independent variables may be used as the training dataset for the predictive model. This may further improve performance of the predictive model, as will be described in more detail below with reference to FIGS. 7 and 8.

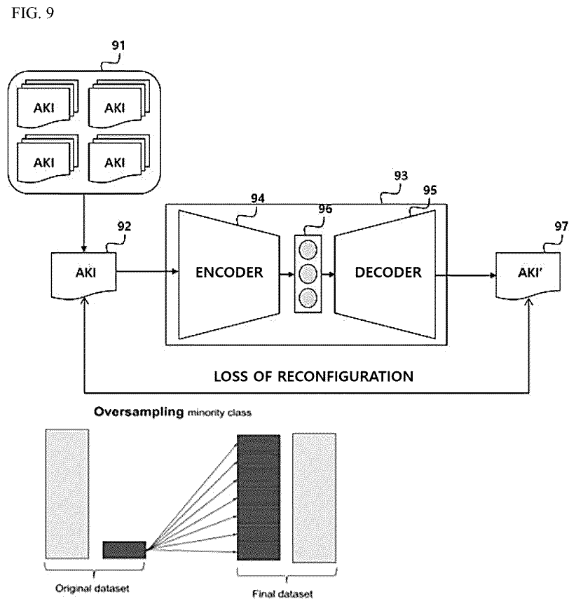

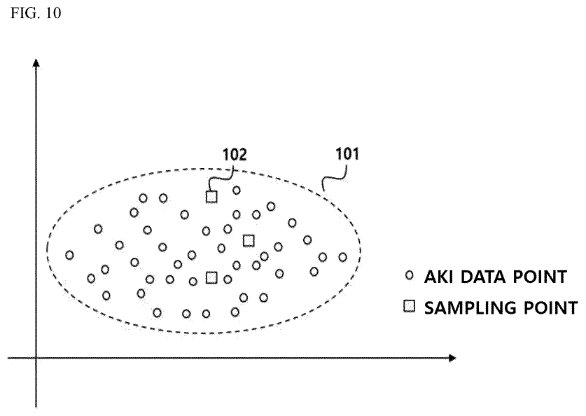

In addition, in some embodiments, a process of augmenting a dataset of an acute kidney injury class (i.e., a group of patients with postoperative acute kidney injury) may be performed to alleviate a class imbalance problem in the original patient dataset (or a patient dataset). This will be described below in more detail with reference to FIGS. 9 and 10.

With reference back to FIG. 4, the description follows.

At step S42, a model for predicting the risk of the occurrence of postoperative acute kidney injury may be built (trained) using a prepared patient dataset. For example, the prediction system 10 may input respective patient data (i.e., values of independent variables) into the predictive model to acquire a prediction result, and train the predictive model in a direction in which a difference (i.e., a prediction error) between the prediction result and the correct answer (i.e., a value of the dependent variable) is minimized.

The predictive model may be designed and implemented based on various types of models. For example, the predictive model may be designed and implemented based on deep learning/machine learning models such as an artificial neural network (see 60 in FIG. 6), logistic regression, light gradient boosting machine (LGBM), naive bayes, support vector machine, decision tree, random forest, and the like. However, the scope of the present disclosure is not limited by these examples, and the predictive model may be implemented based on other types of models (e.g., deep learning models such as a convolutional neural network, recurrent neural network, transformer, etc.). Further, the predictive model may be designed in the form of a classification model (e.g., decision tree, naive bayes, etc.), or may be designed in the form of a regression model.

In some embodiments, multiple predictive models may be built. For example, the prediction system 10 may build a first predictive model (e.g., a predictive model based on an artificial neural network) and further build a second predictive model (e.g., a predictive model based on logistic regression) of a different type from the first predictive model using the prepared patient dataset. Alternatively, the prediction system 10 may build the first predictive model using the patient dataset for first independent variables and build the second predictive model using the patient dataset for second independent variables that are at least partially different from the first independent variables. In this case, the prediction system 10 may predict a risk of the occurrence of postoperative acute kidney injury by comprehensively considering prediction results of the two predictive models.

At step S43, a risk of the occurrence of postoperative acute kidney injury in a specific patient may be predicted using the trained predictive model. For example, the prediction system 10 may predict a risk of the occurrence of postoperative acute kidney injury in a specific patient who has undergone (or is scheduled to undergo) a target surgery through the trained predictive model. Specifically, the prediction system 10 may configure input data for the predictive model based on data of the corresponding patient (e.g., type of the target surgery, duration (or expected duration), preoperative test results, disease history, medication history, etc.), and perform a prediction by inputting the input data to the predictive model. As described above, the predicted results may include, for example, but are not limited to, whether postoperative acute kidney injury occurs, a risk of the occurrence of acute kidney injury (e.g., a confidence score for the acute kidney injury class), a stage of progression/risk of acute kidney injury (e.g., acute kidney injury stage according to the KDIGO guidelines), and a risk for each stage.

With reference to FIGS. 4 to 6, the method of predicting the occurrence of postoperative acute kidney injury has been described according to some embodiments of the present disclosure. As described above, a risk of the occurrence of postoperative acute kidney injury may be predicted early and accurately through machine learning/deep learning models. For example, a risk that acute kidney injury will occur in a patient after surgery may be predicted early and accurately, and an occurrence rate of acute kidney injury may be reduced through proactive actions based on the prediction results.

Hereinafter, embodiments of a method of selecting key independent variables will be described with reference to FIGS. 7 and 8.

First, with reference to FIG. 7, a method for selecting key independent variables according to some embodiments of the present disclosure will be described.

As illustrated in FIG. 7, the present embodiments are directed to a method of selecting key independent variables 73-1 to 73-k based on performance evaluation results for a model 72.

Specifically, the prediction system 10 may train the model 72 using the independent variables 71-1 to 71-n constituting the patient dataset, and evaluate performance for the trained model 72. For example, the prediction system 10 may train the first model using the first independent variable (e.g., 71-1) and train the second model using the second independent variable (e.g., 71-2). Further, the prediction system 10 may evaluate performance of each of the first model and the second model. Of course, the prediction system 10 may also train the model 72 using two or more independent variables (e.g., 71-1 and 71-2).

The model 72 illustrated in FIG. 7 can be understood as an abstraction of all the models used to select the key independent variables. The model 72 is a trainable model (i.e., machine learning/deep learning models), which may be the same type of model as the predictive model described above, or may be a different type of model.

Next, the prediction system 10 may select key independent variables 73-1 to 73-k based on performance evaluation results of the model 72. For example, the prediction system 10 may select K independent variables 73-1 to 73-k (where K is a value less than the total number N of independent variables) that were used to train a model whose performance evaluation score (e.g., accuracy, etc.) is above a reference value as key independent variables.

When the key independent variables 73-1 to 73-k are selected, the prediction system 10 may build the predictive model using the patient dataset that is constituted of the key independent variables 73-1 to 73-k. This enables a higher performance predictive model to be built.

Hereinafter, with reference to FIG. 8, a method for selecting key independent variables will be described according to some other embodiments of the present disclosure.

As illustrated in FIG. 8, the present embodiments are directed to a method of selecting key independent variables using the degree of influence that a change in a value of a specific independent variable (e.g., variable 2) has on the prediction results (e.g., 82 and 83) of a model 81.

Specifically, the prediction system 10 may train the model 81 using the patient dataset. The model 81 is a trainable model (i.e., machine learning/deep learning models), which may be the same type of model as the predictive model described above, or may be a different type of model. Further, the prediction system 10 may input first patient data 84 into the trained model 81 to acquire a first prediction result 82. FIG. 8 illustrates an example where the model 81 is a classification model that outputs confidence scores for an acute kidney injury class (AKI) and a normal class (No-AKI).

Next, the prediction system 10 may change a value 85 of a specific independent variable (e.g., variable 2) in the first patient data 84 to generate second patient data 86, and input the second patient data 86 into the retrained model 81 to acquire a second prediction result 83. For example, the prediction system 10 may change the value 85 of a specific independent variable (e.g., variable 2) to zero, or may change the value 85 to an average value of the dataset belonging to the normal class (or acute kidney injury class).

Next, the prediction system 10 may calculate a difference between the two prediction results 82 and 83. The prediction system 10 may measure the degree of influence (e.g., an average of difference values) that a specific independent variable (e.g., variable 2) has on the prediction results (e.g., 82 and 83) of the model 81 by repeating these processes for other patient data. For example, when changing a value of a specific independent variable (e.g., variable 2) to zero results in a significant overall decrease in the confidence score of the acute kidney injury class (AKI), the prediction system 10 may determine that the independent variable has a high degree of influence on the prediction result (or dependent variable) of the model 81.

The prediction system 10 may measure the degree of influence of each of the independent variables constituting the patient dataset, and select the independent variables whose measured degree of influence is above a reference value as key independent variables. Further, the prediction system 10 may build a predictive model using a patient dataset constituted of key independent variables. This enables a higher performance predictive model to be built.

Meanwhile, according to some other embodiments of the present disclosure, the prediction system 10 may select key independent variables based on odds ratio of the independent variables. Specifically, the prediction system 10 may train a logistic regression model using the patient dataset, and calculate the odds ratio of each of the independent variables from the trained logistic regression model. Then, the prediction system 10 may select the independent variables having an odds ratio that is different from 1 by more than a reference value as key independent variables. For the odds ratios of the independent variables exemplified in Table 1, Table 3 to Table 7 below are referenced.

The embodiments of the method of selecting key independent variables have been described above with reference to FIGS. 7 and 8. Hereinafter, with reference to FIGS. 9 and 10, a method of augmenting a dataset will be described according to some embodiments of the present disclosure.

As illustrated in FIG. 9, the present embodiments are directed to a method of augmenting a patient dataset of the acute kidney injury (AKI) class.

Specifically, the prediction system 10 may sample a latent vector 102 within a data area or latent area 101 in which a patient dataset of the acute kidney injury (AKI) class is encoded. A method of mapping (or encoding) a patient dataset of the acute kidney injury (AKI) class to a data area or latent area may be done in any approach. Further, the prediction system 10 may generate virtual patient data belonging to the acute kidney injury (AKI) class by decoding the sampled latent vector 102. As this sampling and decoding process is repeated, the dataset for the acute kidney injury (AKI) class may be easily augmented.

With reference to FIGS. 9 and 10, the method of augmenting a dataset has been described according to some embodiments of the present disclosure. As described above, by augmenting the patient dataset of the acute kidney injury (AKI) class, the class imbalance problem can be greatly alleviated, and the performance of the predictive model can be significantly improved.

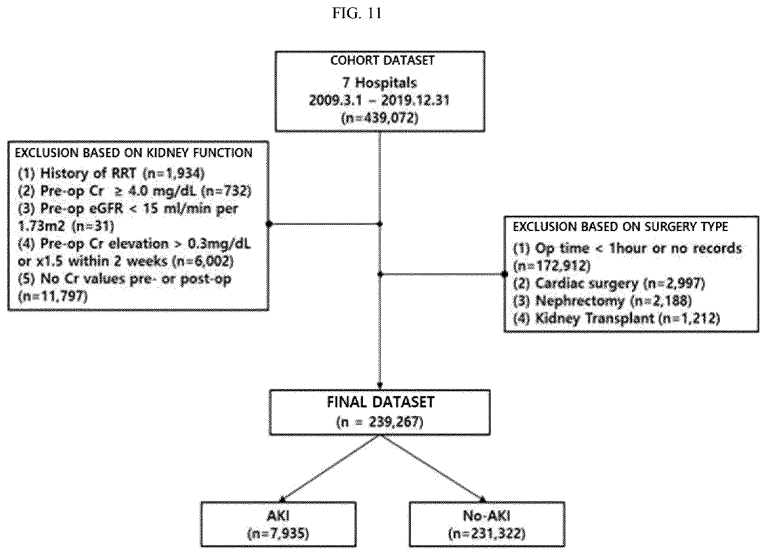

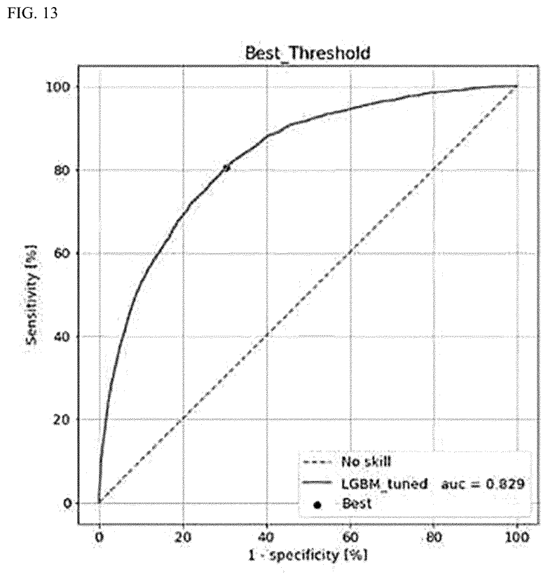

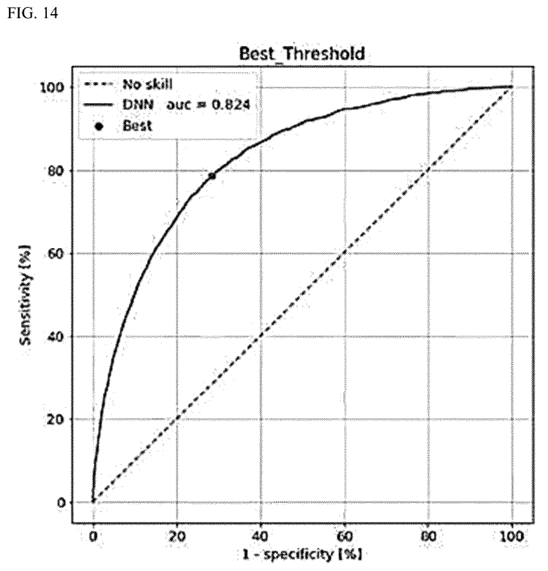

Hereinafter, with reference to FIGS. 11 through 14, results of experiments performed by the inventors of the present disclosure will be briefly described.

To prove the effectiveness of the technical spirit of the present disclosure, the inventors built a model to predict a risk of the occurrence of postoperative acute kidney injury (more precisely, a risk of the occurrence of acute kidney injury within 30 days after surgery) using a dataset of actual patients and evaluated the performance of the built model.

More specifically, as illustrated in FIG. 11, the inventors prepared a final dataset to be used for training and the performance evaluation of the predictive model by cleaning the patient dataset (i.e., cohort dataset) and removing some patient data satisfying the conditions (refer to the description of FIG. 5). The final dataset consisted of a total of 239,267 data (i.e., data sample), of which the number of data corresponding to the acute kidney injury class was 7,935 (i.e., whether acute kidney injury occurred was used as the dependent variable). In addition, the final dataset consisted of patient data for the independent variables exemplified in Table 1.

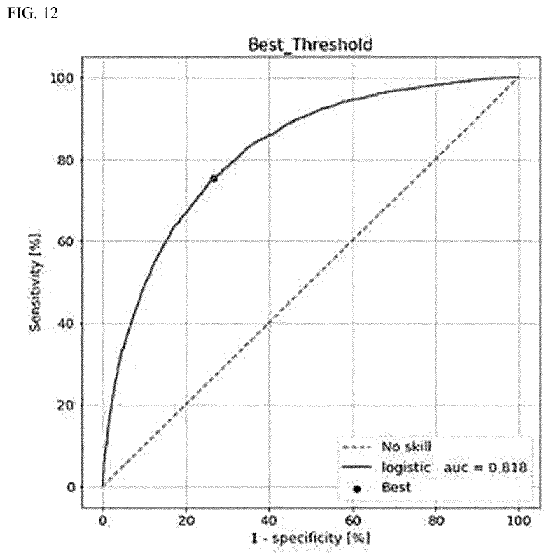

Next, the inventors set about 80% of the final dataset as a training dataset and the remaining about 20% as a test dataset. Further, the inventors built predictive models based on an artificial neural network, logistic regression, decision tree, random forest, LGBM, and naive bayes using the training dataset, and evaluated the performance of each of the predictive models using the test dataset. Results of the evaluation are shown in Table 2 and FIGS. 12 to 14 below. FIGS. 12 to 14 illustrate results of the area under the curve (AUC) evaluation for a logistic regression, artificial neural network, and LGBM, respectively. The evaluation metrics shown in Table 2 and FIGS. 12 to 14 will be well known to those skilled in the art and will not be described herein.

TABLE 2

| | | | | | Sensitivity | F1 − |
|---|---|---|---|---|---|---|
| Classification | AUC | Accuracy | Precision | Specificity | (Recall) | Score |
| Artificial | 0.824 | 0.727 | 0.088 | 0.725 | 0.775 | 0.159 |
| neural | | | | | | |
| network | | | | | | |
| Logistic | 0.818 | 0.735 | 0.089 | 0.735 | 0.754 | 0.159 |
| regression | | | | | | |
| Decision tree | 0.747 | 0.771 | 0.085 | 0.777 | 0.599 | 0.148 |
| Random | 0.803 | 0.704 | 0.081 | 0.702 | 0.764 | 0.146 |
| forest | | | | | | |
| LGBM | 0.828 | 0.712 | 0.085 | 0.709 | 0.793 | 0.154 |

With reference to Table 2 and FIGS. 12 to 14, it can be seen that predictive model based on the artificial neural network perform slightly better, while the performance of other types of predictive models is generally good. It is believed that this is because there are many variables that are closely related to the occurrence of acute kidney injury among the independent variables exemplified in Table 1, and the predictive model performs predictions by considering various factors (independent variables) in a complex manner.

In addition, the inventors calculated an odds ratio of each of the independent variables using a predictive model based on logistic regression to analyze the association of the independent variables with the dependent variable. The calculation results are shown in Table 3 to Table 7 below. Table 3 and Table 4 show the odds ratios of the independent variables for demographic characteristics and disease history, respectively, Table 5 shows the odds ratios of the independent variables for medication history and duration of surgery, and Table 6 and Table 7 show the odds ratios of the independent variables for preoperative examination items.

TABLE 3

| | | 95% confidence | |
|---|---|---|---|
| Classification | Odds ratio | interval | p-value |
| Age | 1.021 | 1.019, 1.023 | <0.0001* |
| Gender | 0.690 | 0.652, 0.730 | <0.0001* |
| BMI | 1.011 | 1.006, 1.017 | 0.0001* |

TABLE 4

| | | 95% confidence | |
|---|---|---|---|
| Classification | Odds ratio | interval | p-value |
| Chronic kidney disease | 2.248 | 1.728, 2.925 | <0.0001* |
| (CKD) | | | |
| Diabetes mellitus (DM) | 1.161 | 1.050, 1.284 | 0.0037* |
| Hypertension (HTN) | 1.210 | 1.080, 1.357 | 0.0011* |
| Cardiovascular disease | 1.217 | 1.118, 1.326 | <0.0001* |
| (CVD) | | | |
| Coronary artery disease | 1.049 | 0.917, 1.199 | 0.4873 |
| (CAD) | | | |
| Chronic obstructive | 1.136 | 0.896, 1.441 | 0.2934 |
| pulmonary disease (COPD) | | | |
| Liver cirrhosis (LC) | 1.316 | 1.086, 1.595 | 0.0051* |

TABLE 5

| | | 95% confidence | |
|---|---|---|---|
| Classification | Odds ratio | interval | p-value |
| Duration of surgery | 1.164 | 1.150, 1.178 | <0.0001* |
| Taking ARB/ACEi | 1.326 | 1.216, 1.447 | <0.0001* |
| Taking NSAIDs | 1.000 | 0.941, 1.062 | 0.9997 |

TABLE 6

| | | 95% confidence | |
|---|---|---|---|
| Classification | Odds ratio | interval | p-value |
| SBP | 1.013 | 1.011, 1.014 | <0.0001* |
| DBP | 0.983 | 0.981, 0.985 | <0.0001* |
| Albumin | 0.524 | 0.489, 0.561 | <0.0001* |
| ALT | 1.000 | 0.999, 1.001 | 0.6259 |
| AST | 1.000 | 1.000, 1.001 | 0.5229 |
| BUN | 1.001 | 0.997, 1.005 | 0.6289 |
| Calcium (Ca) | 1.003 | 0.959, 1.049 | 0.8826 |
| Chlorine (Cl) | 1.005 | 0.997, 1.013 | 0.2040 |
| CPK | 1.000 | 1.000, 1.000 | 0.1227 |
| Creatinine (Cr) | 3.218 | 2.871, 3.607 | <0.0001* |

TABLE 7

| | | 95% confidence | |
|---|---|---|---|
| Classification | Odds ratio | interval | p-value |
| CRP | 0.998 | 0.997, 0.999 | 0.0001* |
| eGFR | 1.012 | 1.011, 1.013 | <0.0001* |
| Glucose | 1.002 | 1.002, 1.002 | <0.0001* |
| Hemoglobin | 1.000 | 0.958, 1.043 | 0.9925 |
| Hematocrit | 0.963 | 0.948, 0.978 | <0.0001* |
| Potassium (K) | 0.755 | 0.715, 0.798 | <0.0001* |
| LDH | 1.000 | 1.000, 1.000 | <0.0001* |
| Sodium (Na) | 0.980 | 0.971, 0.990 | <0.0001* |
| Total protein | 1.042 | 0.998, 1.089 | 0.0644 |
| Uric acid | 1.078 | 1.062, 1.095 | <0.0001* |
| Protein in urine | 1.369 | 1.325, 1.416 | <0.0001* |
| Urinary specific gravity (SG) | 0.042 | 0.009, 0.196 | <0.0001* |
| Urine white blood cell count | 1.005 | 1.003, 1.008 | <0.0001* |
| (WBC) | | | |

Withe reference to Table 3 and Tables 5 to 7, it can be seen that the independent variables regarding gender, chronic kidney disease (CKD), hypertension (HTN), cardiovascular disease (CVD), liver cirrhosis (LC), whether taking an angiotensin receptor blocker (ARB) or angiotensin-converting enzyme inhibitor (ACEi), and non-steroidal anti-inflammatory drugs (NSAIDs), and the like, have a relatively strong association with the dependent variables (e.g., whether postoperative acute kidney injury occurred). In addition, it can be seen that the independent variables regarding albumin, creatinine (Cr), potassium (K), protein, urinary specific gravity (SG), and the like have a relatively strong association with the dependent variables.

The experimental results that the inventors have performed so far have been briefly described. Hereinafter, with reference to FIG. 15, an exemplary computing device 150 capable of implementing the prediction system 10 according to some embodiments of the present disclosure will be described.

FIG. 15 is an exemplary hardware configuration diagram illustrating a computing device 150.

As illustrated in FIG. 15, the computing device 150 may include one or more processors 151, a bus 153, a communication interface 154, a memory 152 that loads computer programs 156 executed by the processors 151, and a storage 155 that stores the computer programs 156. However, FIG. 15 only illustrates the constituent elements that are relevant to the embodiments of the present disclosure. Therefore, it will be appreciated by those of ordinary skill in the art to which the present disclosure belongs that other general purpose constituent elements may be further included in addition to those illustrated in FIG. 15. That is, the computing device 150 may further include various other constituent elements in addition to the constituent elements illustrated in FIG. 15. In addition, the computing device 150 may be configured in the form in which some of the constituent elements illustrated in FIG. 15 are omitted in some cases. Hereinafter, each of the constituent elements of the computing device 150 will be described.

The processor 151 may control an overall operation of each configuration of the computing device 150. The processor 151 may be configured to include at least one of a central processing unit (CPU), a micro processor unit (MPU), a micro controller unit (MCU), a graphic processing unit (GPU), or any other form of processor well known in the art of the present disclosure. In addition, the processor 151 may perform calculations on at least one of the applications or programs for executing the operations/methods according to embodiments of the present disclosure. The computing device 150 may include one or more processors.

Next, the memory 152 may store various data, instructions, and/or information. The memory 152 may load the computer program 156 from the storage 155 to execute the operations/methods according to embodiments of the present disclosure. The memory 152 may be implemented as volatile memory, such as RAM, but the technical scope of the present disclosure is not limited thereto.

Next, the bus 153 may provide a communication function between the constituent elements of the computing device 150. The bus 153 may be implemented as various types of buses, such as an address bus, data bus, and control bus.

Next, a communication interface 154 may support wired and wireless Internet communication of the computing device 150. In addition, the communication interface 154 may support a variety of communication methods in addition to Internet communication. To this end, the communication interface 154 may be configured to include a communication module well known in the art of the present disclosure.

Next, the storage 155 may non-temporarily store one or more computer programs 156. The storage 155 may be configured to include non-volatile memory, such as read only memory (ROM), erasable programmable ROM (EPROM), electrically erasable programmable ROM (EEPROM), flash memory, or the like, a hard disk, a removable disk, or any other form of computer-readable recording medium well known in the art of the present disclosure.

Next, the computer program 156 may include one or more instructions that allow the processor 151 to perform operations/methods according to various embodiments of the present disclosure when loaded into memory 152. That is, the processor 151 may perform operations/methods according to various embodiments of the present disclosure by executing the one or more instructions.

For example, the computer program 156 may include one or more instructions to allow an operation to acquire a trained model to predict a risk of the occurrence of postoperative acute kidney injury, and an operation to predict a risk of the occurrence of acute kidney injury to a patient after a target surgery using the trained model. In this case, the prediction system 10 according to some embodiments of the present disclosure may be implemented through the computing device 150.

With reference to FIG. 15, the exemplary computing device 150 capable of implementing the prediction system 10 according to some embodiments of the present disclosure has been described.

The technical spirit of the present disclosure that has been described with reference to FIGS. 1 to 15 may be implemented as a computer-readable code on a computer-readable medium. The computer-readable recording medium may be, for example, a portable recording medium (CD, DVD, Blu-ray disk, USB storage device, or portable hard disk) or a fixed recording medium (ROM, RAM, or computer-installed hard disk). The computer program recorded on the computer-readable recording medium may be transmitted to different computing device through a network, such as the Internet, and installed on the different computing device, and thereby used on the different computing device.

While all of the constituent elements that constitute the embodiments of the present disclosure have been described above as being combined or operating in combination, the technical spirit of the present disclosure are not necessarily limited to the embodiments. That is, one or more of the constituent elements may be selectively combined and operated within the object of the present disclosure.

Although the operations are illustrated in a specific order in the drawings, it should not be understood that the operations must be executed in the specific order illustrated or in sequential order, or that all of the illustrated operations must be executed to achieve the desired result. In a specific situation, multitasking and parallel processing may be advantageous. Moreover, it should be understood that the separation of the various configurations in the embodiments described above is not necessarily intended to imply that such separation is required, and that the program components and systems described may generally be integrated together into a single software product or packaged into multiple software products.

While the exemplary embodiments of the present disclosure have been described with reference to the accompanying drawings, those skilled in the art will understand that the present disclosure may be carried out in any other specific form without changing the technical spirit or an essential feature thereof. Therefore, it should be understood that the above-described exemplary embodiments are illustrative in all aspects and do not limit the present invention. The protective scope of the present disclosure should be construed based on the following claims, and all the technical spirit in the equivalent scope thereto should be construed as falling within the scope of the technical spirit defined by the present invention.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of predicting an occurrence of acute kidney injury, which is performed by at least one computing device, comprising:
preparing a dataset of a plurality of patients—wherein a dependent variable of the dataset relates to an occurrence of postoperative acute kidney injury, and independent variables of the dataset include variables relating to preoperative examination items of the patients—; and
building a model configured to predict a risk of the occurrence of postoperative acute kidney injury using the prepared dataset.

2. The method of claim 1, wherein the preoperative examination items comprise albumin, creatinine (Cr), potassium, protein, and urinary specific gravity.

3. The method of claim 1, wherein the independent variables of the dataset further comprise variables relating to disease history and medication history of the patients,
wherein the disease history comprises history of chronic kidney disease (CKD), hypertension (HTN), cardiovascular disease (CVD), chronic obstructive pulmonary disease (COPD), and liver cirrhosis (LC), and
wherein the medication history relates to antihypertensive drugs.

4. The method of claim 1, wherein the independent variables of the dataset further comprise variables regarding types and duration of surgeries undergone by the patients.

5. The method of claim 1, wherein the model is based on at least one of a neural network, logistic regression, and a light gradient boosting machine (LGBM).

6. The method of claim 1, wherein the preparing of the dataset comprises removing a patient data satisfying a predetermined kidney-related condition from an original patient dataset.

7. The method of claim 6, wherein the predetermined kidney-related condition is defined based on history of renal replacement therapy or a preoperative eGFR value.

8. The method of claim 6, wherein the predetermined kidney-related condition is defined based on a preoperative creatinine (Cr) level or a degree of elevation of the creatinine (Cr) level within a predetermined period of time prior to surgery.

9. The method of claim 1, wherein the preparing of the dataset comprises removing patient data satisfying a predetermined surgery-related condition from an original patient dataset, and
wherein the predetermined surgery-related condition is defined based on duration of surgery or types of surgeries.

10. The method of claim 1, wherein the preparing of the dataset comprises:
correcting for outliers in the original patient dataset;
correcting for missing values in the original patient dataset using multiple imputation by chained equations; and
normalizing the original patient dataset corrected for the outliers and the missing values.

11. The method of claim 1, wherein the preparing of the dataset comprises:
acquiring an original patient dataset—wherein the original patient dataset includes a first dataset for a patient group that has an occurrence of postoperative acute kidney injury and a second dataset for a patient group that does not have an occurrence of postoperative acute kidney injury—; and
augmenting the first dataset.

12. A method of predicting an occurrence of acute kidney injury, which is performed by at least one computing device, comprising:
acquiring a model trained to predict a risk of an occurrence of postoperative acute kidney injury—wherein the model is trained using a dataset of a plurality of patients, a dependent variable of the dataset is the occurrence of postoperative acute kidney injury, and independent variables of the dataset include variables relating to preoperative examination items of the patients; and
predicting a risk of an occurrence of acute kidney injury to a specific patient after a target surgery using the trained model.

13. The method of claim 12, wherein the independent variables of the dataset further comprise variables regarding types and durations of surgeries undergone by the patients, and
wherein the predicting comprises:
constituting input data based on a type and duration of the target surgery, and examination results of the specific patient for the preoperative examination items; and
predicting the risk by inputting the input data into the trained model.

14. A system for predicting an occurrence of acute kidney injury comprising:
one or more processors; and
a memory configured to store one or more instructions,
wherein the one or more processors perform:
acquiring, by executing the stored one or more instructions, a model trained to predict a risk of an occurrence of postoperative acute kidney injury—wherein the model is trained using a dataset of a plurality of patients, a dependent variable of the dataset relates to the occurrence of postoperative acute kidney injury, and independent variables of the dataset include variables relating to preoperative examination items of the patients; and
predicting a risk of an occurrence of acute kidney injury to a patient after a target surgery using the trained model.
